(19) United States
(12) Patent Application Publication
(10) Pub. No.: US 2005/0175499 A1
(43) Pub. Date: Aug. 11, 2005

(54) Method of reducing cross sample contamination during filter sampling

(75) Inventors: Michael A. Lewinski, San Clemente, CA (US); William A. Meyer III, Catonsville, MD (US)

Correspondence Address: FOLEY & LARDNER, P.O. BOX 80278, SAN DIEGO, CA, 92138-0278, US

(73) Assignee: Quest Diagnostics Investments Incorporated

(21) Appl. No.: 10/773,752

(22) Filed: Feb. 6, 2004

(51) Int. Cl. A61L 2/16

(52) U.S. Cl. 422/28

(57) ABSTRACT

Provided are methods for reducing transfer of fluid between liquid samples subject to consecutive instrument controlled filter sampling. Transfer of fluid between subsequent samples is reduced by decontaminating surfaces of the instrument. The methods reduce sample contamination so that any remaining unused sample may be subject to diagnostic analysis without less concern that the outcome will be the result of sample cross-contamination.

### FIELD OF THE INVENTION

The present invention relates generally to methods of reducing cross contamination from one sample to a successive sample where both samples are subject to filtration.

### BACKGROUND OF THE INVENTION

The following discussion of the background of the invention is merely provided to aid the reader in understanding the invention and is not admitted to describe or constitute prior art to the present invention.

Instruments that process different samples may be subject to cross-contamination unless the instrument is suitable designed or properly disinfected. Cross-contamination from a previously run sample to a subsequently run sample may occur and involve materials naturally present in the sample or foreign agents or components thereof, which are not normally present in the sample. For example, a sample containing a pathogenic bacteria or containing biological material evidencing the organism or agent (e.g., nucleic acid or protein) can be the source of sample cross-contamination when processing different samples through the same instrument.

Cross-contamination is particularly problematic when utilizing highly sensitive detection methods. For example, cross-contamination in the Polymerase Chain Reaction (PCR) technique is a vexing problem that is addressed to some extent by the use of disposable materials for sample handling (e.g. disposable gloves, vials, pipette tips etc.) and by processes for decontaminating surfaces. See Cone et al., PCR Methods Appl. (1993) (3):S15-7 (using ultraviolet light); Deragon et al., Nucleic Acids Res. (1990) 18(20):6149 (using gamma irradiation); Padua et al., Leukemia (1999) 13(11):1898-9 (using ultraviolet light and clean air).

### BRIEF SUMMARY OF THE INVENTION

In a first aspect, the invention relates to methods of reducing contamination involving successively processed liquid samples. In one embodiment, the present invention provides methods of reducing transfer of fluid between liquid samples subject to consecutive instrument controlled filter sampling.

In one embodiment, the method comprises a) drawing fluid from a sample vial containing a first liquid sample through a first filter using the instrument. The filter is removed from the instrument and replaced with a new filter. Before and/or after this step, surfaces of the instrument are decontaminated to remove first sample material released as an aerosol by filter rotation or inversion or dropped by gravity flow from the filter. Finally, the instrument is used to draw fluid from a sample vial containing a second liquid sample through the second filter. By decontaminating the instrument surfaces, less first liquid sample is available to contaminate the second liquid sample that remains in the second sample vial. Thus, because the remaining unfiltered second sample in the vial is less contaminated, it can be subjected to methods of diagnostic evaluation, such as nucleic acid amplification and detection, with greater confidence that results are not affected by sample cross contamination.

In another embodiment, the filter is contained within the instrument and mounted at one end of a tube shaped hollow element and wherein the other end of the tube shaped hollow element is mounted to and in fluid communication with a filter cap assembly, which functions to fluidly link the filter to a pressure source.

In one embodiment, decontamination is achieved by wiping the instrument surfaces with an absorbent material. In another embodiment, decontamination is achieved by applying a decontamination solution to the instrument surfaces. Preferably, decontamination is applied to the filter cap assembly.

In yet another embodiment, the instrument surfaces are in the same compartment of the instrument as the filter.

In still yet another embodiment, the instrument used for filtration is a ThinPrep® 2000 Processor or similar such instrument.

In a second aspect, the invention relates to methods of reducing transfer of nucleic acid between liquid samples subjected to consecutive instrument controlled filter sampling.

In one embodiment, the method comprises a) drawing fluid from a sample vial containing a first liquid sample through a first filter using the instrument. The filter is removed and replaced with a new filter. Before and/or after this step, surfaces of the instrument are decontaminated to remove, denature or destroy nucleic acid from the first sample released as aerosol by filter rotation or inversion or dropped by gravity flow from the filter. Finally, the instrument is used to draw fluid from a sample vial containing second liquid sample through the second filter. By decontaminating the first sample fluid from the surfaces of the instrument, less sample nucleic acid that may cause contamination is available to contaminate the second liquid sample that remains in the second sample vial. Thus, because the remaining unfiltered second sample in the vial is less contaminated, it can be subjected to methods of nucleic acid amplification and detection with greater confidence that amplified products were not the result of sample cross-contamination.

The decontaminating solution may be a disinfectant such as bleach.

In another embodiment, the filter is contained within the instrument and mounted at one end of a tube shaped hollow element and wherein the other end of the tube shaped hollow element is mounted to and in fluid communication with a filter cap assembly, which functions to fluidly link the filter to a pressure source.

In one embodiment, decontamination is achieved by wiping the instrument surfaces with an absorbent material. In another embodiment, decontamination is achieved by applying a decontamination solution to the instrument surfaces. Preferably, decontamination is applied to the filter cap assembly.

In yet another embodiment, the instrument surfaces are in the same compartment of the instrument as the filter.

In still yet another embodiment, the instrument used for filtration is a ThinPrep® 2000 Processor or similar such instrument.

### BRIEF DESCRIPTION OF THE FIGURES

FIG. 1 shows a photograph of the front of the ThinPrep® 2000 Processor with the door closed. A removable filter (clear) is seen as the small object at far right fits onto a filter assembly. The removable filter is a tube with the filter on one end and the other end open.

FIG. 2 shows a photograph of the front of the ThinPrep® 2000 Processor with the door open, exposing the filter cap assembly (identified) and the filter mounted in place. Below the filter is a sample vial containing a liquid patient sample. The filter cap assembly manifold with attached tubing is located directly above the filter cap assembly. A microscope slide is held in place in the assembly at the upper part of the instrument.

### DETAILED DESCRIPTION OF THE PREFERRED EMBODIMENTS

The present invention provides methods of reducing cross-contamination between liquid samples. Cross-contamination as used herein refers to contamination one way from one sample to a subsequent sample.

In one embodiment, the present invention provides methods of reducing transfer of fluid between liquid samples subject to consecutive instrument controlled filter sampling. In another embodiment, the present invention provides methods of reducing transfer of nucleic acid between liquid samples subjected to consecutive instrument controlled filter sampling.

In accordance with one embodiment of this method, a sample vial containing a first liquid sample is placed into the instrument and fluid from the sample is drawn through a first filter using an automated instrument. The first filter is discarded and a second filter is loaded in its place. Before and/or after the filter change, surfaces of the instrument are decontaminated so as to remove first sample released as aerosol by filter rotation or inversion filter or dropped by gravity flow from the filter. A second sample vial containing a second liquid sample is placed into the instrument and fluid from the second sample is drawn through a second filter using the instrument. By decontaminating the instrument surfaces, the amount of the first sample that contaminates any remaining portion of the second liquid sample in the second sample vial is reduced. In one embodiment, the step of decontaminating includes wiping with a liquid absorbent material. In another embodiment, the step of decontaminating involves applying a decontamination solution to the instrument surfaces.

In one embodiment, the filter is contained within the instrument and mounted at one end of a tube shaped hollow element; the other end of the tube shaped hollow element is mounted to and in fluid communication with a filter cap assembly. In another embodiment, the filter cap assembly is fluidly connected with a manifold, which is linked to a negative and/or positive pressure source. Thus, in this latter embodiment, the pressure source is linked so that pressure can be applied to the filter to draw liquid from the sample and through the filter. In a further embodiment, the instrument surfaces are in the same compartment of the instrument as the filter.

As used herein, the term “reducing transfer of a first liquid sample subjected to filtration to the remainder of a subsequent liquid sample, a portion of which was filtered by the same instrument” means that the amount of the first liquid that results in contaminating the second liquid sample in the second sample vial is reduced from what it would have been in the absence of decontaminating the instrument surfaces. Because any remaining second liquid sample in the second sample vial following completion of the filtration step is less contaminated, this residual sample may be subjected to diagnostic analysis such as nucleic acid amplification to detect a microorganism in the sample or determine an individual's genotype or phenotype with more confidence that the results do not reflect cross sample contamination.

Nucleic acid amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. Jun. 1, 2000 ;29(11):E54-E54; Hafner et al., Biotechniques Apr. 30, 2001(4):852-6, 858, 860; and Zhong et al., Biotechniques Apr. 30, 2001(4):852-6, 858, 860. These methods increase the representation of a population of nucleic acid sequences in a sample to facilitate their detection. Amplification can be achieved with oligonucleotide primers which are short polymers composed of deoxyribonucleotides, ribonucleotides or any combination thereof. Oligonucleotides are at least 9 nucleotides in length, preferably 20 to 70 nucleotides long, with 21 to 26 nucleotides being the most common. In certain embodiments, the oligonucleotides are joined together with a detectable label. Oligonucleotide probes which may be detectably labeled can be used to detect amplified nucleic acid using the principle of nucleic acid hybridization, a process whereby two complementary nucleic acid strands anneal to each other under appropriately stringent conditions. See, e.g., Sambrook, et al., 1989, Molecular Cloning: A Laboratory Manual, Second Edition, Cold Spring Harbor Press, Plainview, N.Y.

As used herein, the term “drawing fluid from a (first or second ) sample vial containing a (first or second, respectively) liquid sample through a (first or second, respectively) filter using the instrument” refers to the process where the instrument applies a negative or positive pressure to the filter, thereby drawing (or driving) liquid sample through the filter. With cell containing samples, the filter is chosen with a sufficient pore size that prevents some or all the cells from passing through the filter.

As used herein, the term “decontaminating surfaces of the instrument so as to remove first sample” refers to removal, denaturing, or destruction of contaminating sample from the instrument surfaces. As used herein, instrument surfaces includes the outside of the instrument, the inside of the instrument and parts of the instrument such as the filter cap assembly. In one embodiment, decontamination is achieved by contacting or wiping instrument surfaces with an absorbent material. Any absorbent material can be used such as gauze, a paper towel, kimwipe, Biowipe, and the like. A “biowipe,” which has an absorbent surface on one side and a liquid impermeable surface such as latex on the other side is a useful absorbent material. First liquid sample that contaminates surfaces of the instrument may be unfiltered or filtered by the instrument.

As used herein, the term “instrument surfaces are in the same compartment of the instrument as the filter” refers to various surfaces of the instrument that may be decontaminated by sample liquid caused by sample aerosol resulting from mixing the sample using the filter and/or by dripping sample from the filter.

As used herein, the term “decontaminating surfaces of the instrument so as to remove, denature or destroy nucleic acid” refers to contacting surfaces of the instrument with an absorbent material or with a decontaminating solution. Contacting with a decontaminating solution may be achieved immersion such as in the case of a filter cap assembly or by wiping with an absorbent material to which decontaminating solution has been applied. The surfaces to be decontaminated with solution also may be on the outside of the instrument.

As used herein, the term “decontaminating solution” refers to any solution that is effective in disinfecting, denaturing or otherwise destroying organisms and/or biological molecules (e.g., nucleic acid, protein, etc.) that may be present in a liquid sample and which have contaminated a surface of the instrument. A preferred decontaminating solution is a solution of about 0.5% sodium hyperchlorite solution in water (i.e., 10% household bleach).

As used herein, the term “sample” refers to any liquid containing sample, which preferably includes cells. Such cell containing liquid sample may include a blood containing sample, a cervical cells containing sample, a fine needle biopsy containing sample, and the like. Cells may be obtained from a biological source such an animal tissue or animal fluid such as lymphatic fluid, cerebrospinal fluid, synovial fluid, urine, saliva, mucous membranes, and the like. A human sample with human cells is a preferred sample. The term “patient sample” as used herein refers to a sample obtained from a human seeking diagnosis or treatment of a disease.

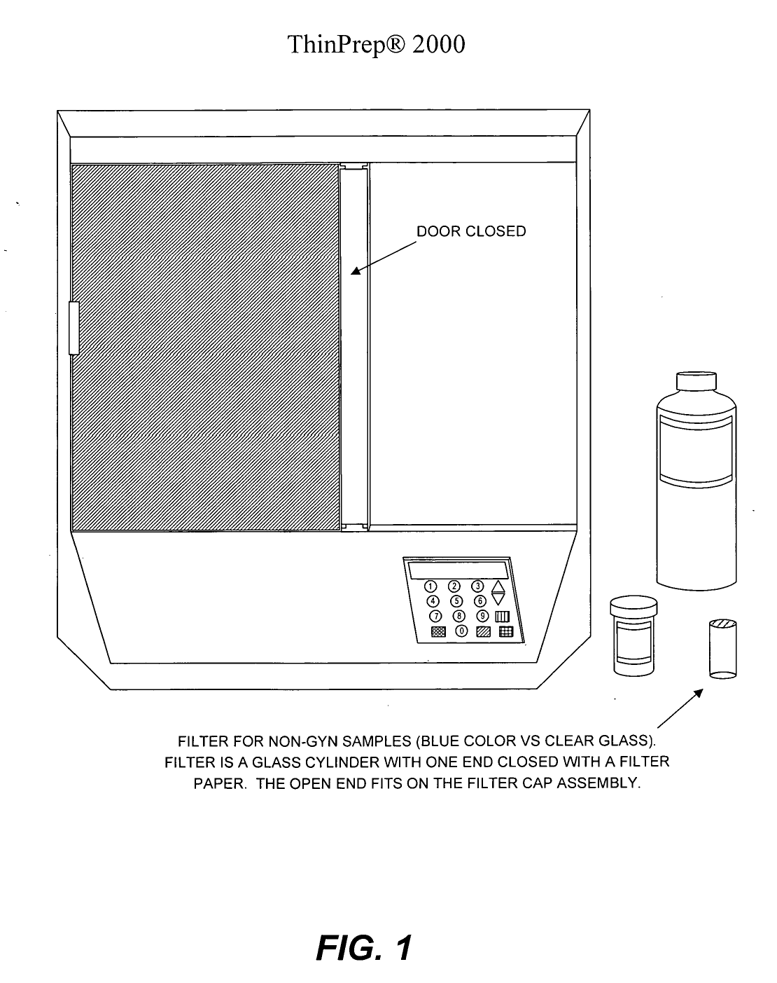

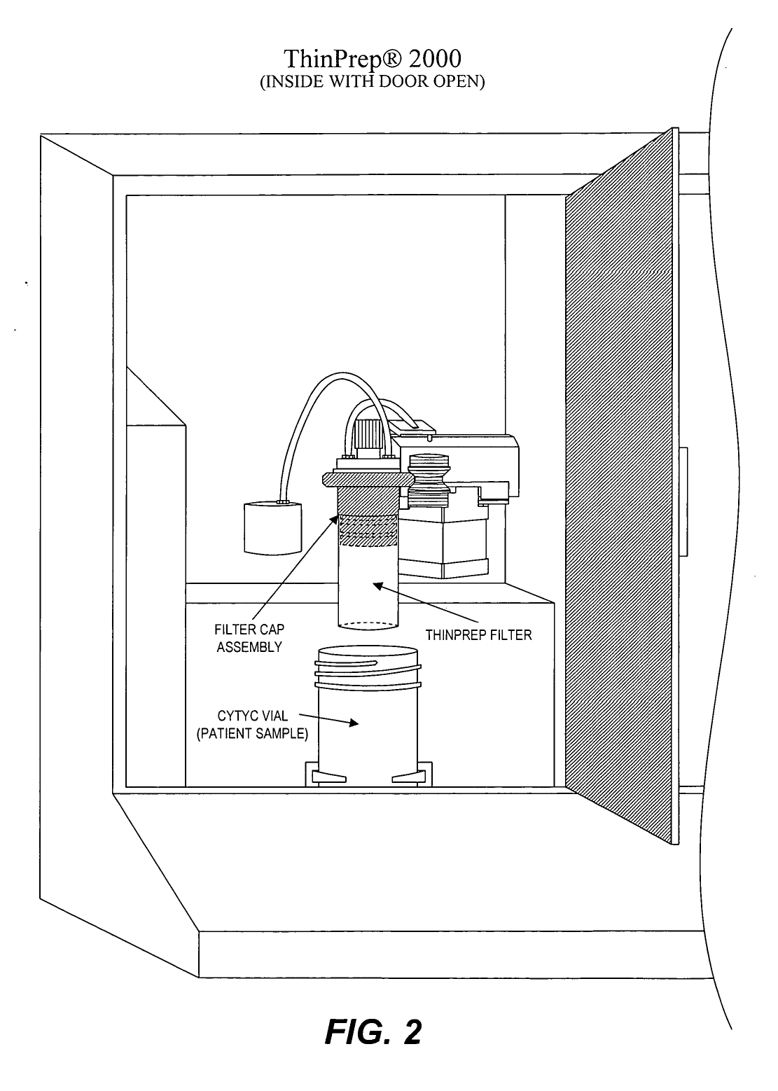

A embodiment of a filter sampling instrument that is suitable for use in the methods of the present invention is the ThinPrep® 2000 Processor, an automated slide preparation unit used for separating cells from fluid for microscopic analysis. The device is manufactured by Cytyc Corporation, Marlborough, Mass. U.S.A. FIG. 1 shows a front view of the instrument with the doors closed and FIG. 2 shows the inside of the instrument exposing the filter cap assembly and ThinPrep® filter.

The ThinPrep 2000 consists of four major components:
1. Mechanics for holding and moving the sample vial, the filter and the microscope slide during instrument processing.
2. Pneumatics for generating controlled positive and negative pressure at the filter.
3. Electronics to power the motors, valves and sensors that make up the mechanics and pneumatics; and
4. Hardware and software to orchestrate the instrument components and processes.

Instrument specifications from the manufacturer are as follows:

Throughput:

Approximately 25 samples per hour. Up to 50,000 samples per year with a single 8-hour shift.

Dimensions:

18″ wide×15″ deep×19.5″ high

46 cm×38 cm×50 cm

approximately 41 lb/18.6 kg

Power Requirements:

Voltage Autoranging between 100/120 and 220/240 VAC±10%.

The ThinPrep filter is a flat disc located at one end of a hollow tube. The other end of the tube is open. The filter and tube in which the filter is embodied will be referred to collectively as the filter. The pore size of the filter can vary with the type of sample to be collected. A preferred filter has a suitable pore size and/or pore shape to filter out one or more cell types from the sample. Sample may include blood, cervical cell scrapings (i.e., Pap smears), fine needle biopsies and the like. Use of the ThinPrep® 2000 to prepare slides using fine needle aspiration biopsy material has been described previously. Dey et al., Acta Cytol. (2000) 44(1):46-50.

The filter cap assembly is a hollow tube shaped element with one end having a larger circumference than the other end. The small circumference end of the filter cap assembly contains a pair of circular O-rings located on the outside (FIG. 2). The inside circumference of the open end of the filter fits snugly over the end of the filter cap assembly that contains the O-rings so that the O-rings seal against the inside of the filter. The larger circumference end of the filter cap assembly attaches to a manifold, which is fixedly mounted into the instrument. Thus, the filter is contained within the instrument and mounted at one end of a tube shaped hollow element, the other end of the tube shaped hollow element being mounted to and in fluid communication with the filter cap assembly, which functions to fluidly link the filter to a pressure source.

The manufacturer recommends collecting patient specimens in a sample vial containing a methanol-buffer containing preservative, PreservCyt® solution. An agent that lyses red blood cells may be included in the preservative if samples are known to contain red blood cells. The opening of the sample vial is large enough so that the filter can be inserted directly into the sample vial and contact the liquid sample. Cell containing liquid samples may be prepared using other liquid solutions well known in the art such as a buffered saline solution.

During operation of the ThinPrep° 2000 Processor, the operator removes the reusable filter cap assembly and attaches a new ThinPrep® filter. The operator then attaches the filter cap assembly/filter to the instrument by mounting to the manifold. In this embodiment, various instrument surfaces (e.g. the outside surface of the filter cap assembly) are in the same compartment of the instrument as the filter.

The operator then removes the cover from the specimen vial and sets the specimen vial below the filter/filter cap assembly in the instrument. The instrument door is closed and the start button on the instrument is pressed. The filter is automatically lowered into the open vial and is spun at high rpm's to mix the sample. A series of negative pressure pulses are generated which draw fluid though the filter to collect a thin, even layer of diagnostic cellular material on the filter. The ThinPrep® 2000 Processor constantly monitors the rate of flow through the ThinPrep Filter during this process and stops drawing fluid once the filter begins to clog. The instrument inverts the manifold/filter cap assembly/filter and presses the end of the filter with accumulated cellular against a glass slide to create an impression smear. The slide is then ejected into a cell fixative bath, ready for staining and evaluation.

It has been surprisingly found that the use of disposable filters and disposable gloves changed between samples in the ThinPrep® 2000 Processor results in significant contamination between successive samples; Sample transfer to successive samples occurred 62% of times when observed when no decontamination treatment was employed. Cross-contamination may result from the mixing step prior to sample update into the filter. Mixing disperses patient sample as micro- and macroscopic drops onto various internal surfaces of the instrument including the filter cap assembly (i.e., aeroselization). Another possible source of cross-contamination is the step after the sample vial has been removed from the instrument and before the manifold/filter cap assembly/filter has been inverted. In this case, residual patient sample on the exterior of the filter may fall by gravity and contaminates the instrument floor. Additional fluid in the filter interior may exacerbate this problem. Another source of cross-contamination may occur when the manifold/filter cap assembly/filter is inverted. Residual unfiltered patient sample on the exterior of the filter falls may fall by gravity and contaminate the exterior of the filter cap assembly.

As used herein, “reducing transfer” from a first sample to a second sample in accordance with the invention methods means a reduction of at least 40% in the rate of successive cross-contamination. The reduction in the successive contamination rate is preferably at least rate 50%, more preferably at least 60%, yet more preferably at least 70%, still yet more preferably at least 80%, more preferably at least 90%, more preferably at least 95%, more preferably at least 99% and most preferably 100%. In addition to decontaminating surfaces of the instrument, reduction in sample cross-contamination may be achieved by decontaminating the outside of the instrument and the area around the instrument. Changing gloves between samples also may further reduce cross-contamination.

The following examples serve to illustrate the present invention. These examples are in no way intended to limit the scope of the invention.

### EXAMPLES

Studies were performed to determine the contamination potential when using Cytyc ThinPrep (TP) vials for CT/NG (Chlamydia trachomatis/Neisseria gonorrhoeae) PCR after the vial had been processed for Pap slide preparation. Experiments were performed using 50 alternating, simulated TP positive specimens containing CT infected HEp-2 cells and NG cells and 50 negative TP vials with uninfected HEp-2 cells. Initial results performed without application of any of the invention decontamination procedures using standard Cytyc procedures with the Cytyc ThinPrep® 2000 Processor resulted in 31/50 (62%) positive or equivocal PCR results from negative vials.

Using, plastic backed absorbent biowipes to handle the vials during Cytyc processing and changing gloves between each vial, the contamination rate was reduced to 16%. Use of biowipes, glove changes and wiping down the Cytyc instrument with 10% bleach solution resulted in 36% contamination of CT/NG negative vials. Aliquots removed from the negative vials before Pap slide preparation were all negative for CT/NG by PCR indicating that contamination occurs during Pap smear slide preparation as part of the operation of the Cytyc ThinPrep® 2000 Processor and not during PCR sample preparation or testing on the Roche PCR COBAS instrument.

Use of a “blank” TP vial in-between each of the CT/NG positive and negative vials in three experiments reduced contamination to 0-10%. Further examination of the ThinPrep® 2000 Processor revealed that the filter cap, which holds the removable filter in place, becomes wet with patient sample during routine operation implicating it as a source of specimen-to-specimen cross-contamination. Experiments in which the Cytyc instrument filter cap was removed between each TP sample, immersed in 10% bleach, rinsed in distilled water, and then dried resulted in 0% contamination.

It is concluded that the Cytyc instrument filter cap should be bleached between each sample vial if residual sample in the vial is to be used after Pap slide preparation for amplified CT/NG testing.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are set forth within the following claims.

1. A method of reducing transfer of a first sample subjected to filtration to the remainder of a subsequent sample, a portion of which was filtered by the same instrument, said method comprising:
a) drawing fluid from a first sample vial containing a first sample through a first filter using the instrument,
b) decontaminating surfaces of the instrument so as to remove first sample released as aerosol by the filter or dropped by gravity flow from the filter;
c) removing the first filter and replacing with a second filter;
d) drawing fluid from a second sample vial containing a second sample through the second filter using the instrument,
whereby the step of decontaminating reduces transfer of the first sample to any remaining portion of the second sample in the second sample vial.

2. The method of claim 1 wherein decontamination is achieved by wiping with a liquid absorbent material.

3. The method of claim 1 wherein decontaminating is achieved by contacting the surface with liquid decontaminating solution.

4. The method of claim 3 wherein said liquid decontaminating solution is a disinfectant.

5. The method of claim 4 wherein the disinfectant is a solution of bleach.

6. The method of claim 1 wherein the filter is contained within the instrument and mounted at one end of a tube shaped hollow element and wherein the other end of the tube shaped hollow element is mounted to and in fluid communication with a filter cap assembly which functions to fluidly link the filter to a pressure source.

7. The method of claim 1 wherein the said instrument surfaces are in the same compartment of the instrument as the filter.

8. The method of claim 1 wherein surfaces of the filter cap assembly are decontaminated.

9. The method of claim 1 wherein the instrument is a ThinPrep® 2000 Processor or similar such instrument.

10. The method of claim 1 wherein after step d), the remainder of the second sample in the second sample vial is subject to nucleic acid amplification.

11. The method of claim 1 wherein the first or second sample is selected from the group consisting of a blood containing sample, a cervical cells containing sample, and a fine needle biopsy containing sample.

12. A method of reducing transfer of a first sample subjected to filtration by a ThinPrep® 2000 Processor instrument or similar such instrument to the remainder of a subsequent sample, a portion of which was filtered by the same instrument, said method comprising:
a) drawing fluid from a first sample vial containing a first sample through a first filter using the instrument,
b) decontaminating surfaces of the instrument so as to remove first sample released as aerosol by the filter or dropped by gravity flow from the filter;
c) removing the first filter and loading a as second filter into the instrument; and
d) drawing liquid fluid from a second sample vial containing a second sample through the second filter using the instrument
whereby the step of decontaminating reduces transfer of the first sample to any remaining portion of the second sample in the second sample vial.

13. The method of claim 12 wherein decontamination is achieved by wiping with a liquid absorbent material.

14. The method of claim 12 wherein decontaminating is achieved by contacting the surface with liquid decontaminating solution.

15. The method of claim 14 wherein said liquid decontaminating solution is a disinfectant.

16. The method of claim 15 wherein the disinfectant is a solution of bleach.

17. The method of claim 12 wherein the instrument contains a filter cap assembly which is subjected to decontamination.

18. The method of claim 12 wherein after step d) the remainder of the second sample in the second sample vial is subjected to nucleic acid amplification.

19. The method of claim 12 wherein the sample is selected from the group consisting of a blood containing sample, a cervical cells containing sample, and a fine needle biopsy containing sample.

20. A method of reducing transfer of nucleic acid from a first sample subjected to filtration to the remainder of a subsequent sample, a portion of which was filtered by the same instrument, said method comprising:
a) drawing fluid from a first sample vial containing a first sample through a first filter using the instrument,
b) decontaminating surfaces of the instrument so as to remove, denature or destroy nucleic acid from the first sample released as aerosol by the filter or dropped by gravity flow from the filter;
c) removing the first filter and replacing with a second filter;
d) drawing fluid from a second sample vial containing a second sample through the second filter using the instrument,
whereby the step of decontaminating reduces the amount of nucleic acid transferred from the first sample to any remaining portion of the second sample in the second sample vial.

21. The method of claim 20 wherein decontamination is achieved by wiping with a liquid absorbent material.

22. The method of claim 20 wherein decontaminating is achieved by contacting the surface with liquid decontaminating solution.

23. The method of claim 22 wherein said liquid decontaminating solution is a disinfectant.

24. The method of claim 23 wherein the disinfectant is a solution of bleach.

25. The method of claim 20 wherein the filter is contained within the instrument and mounted at one end of a tube shaped hollow element and wherein the other end of the tube shaped hollow element is mounted to and in fluid communication with a filter cap assembly which functions to fluidly link the filter to a pressure source.

26. The method of claim 20 wherein the said instrument surfaces are in the same compartment of the instrument as the filter.

27. The method of claim 20 wherein surfaces of the filter cap assembly are decontaminated.

28. The method of claim 20 wherein the instrument is a ThinPrep® 2000 Processor or similar such instrument.

29. The method of claim 20 wherein after step d), the remainder of the second sample in the second sample vial is subject to nucleic acid amplification.

30. The method of claim 20 wherein the first or second sample is selected from the group consisting of a blood containing sample, a cervical cells containing sample, and a fine needle biopsy containing sample.

31. A method of reducing transfer of nucleic acid from a first sample subjected to filtration by a ThinPrep® 2000 Processor or similar such instrument to the remainder of a subsequent sample, a portion of which was filtered by the same instrument, said method comprising:
a) drawing fluid from a first sample vial containing a first sample through a first filter using the instrument,
b) decontaminating surfaces of the instrument so as to remove, denature or destroy nucleic acid from the first sample released as aerosol by the filter or dropped by gravity flow from the filter;
c) removing the first filter and loading a as second filter into the instrument; and
d) drawing liquid fluid from a second sample vial containing a second sample through the second filter using the instrument
whereby the step of decontaminating reduces the amount of nucleic acid transferred from the first sample to any remaining portion of the second sample in the second sample vial.

32. The method of claim 31 wherein decontamination is achieved by wiping with a liquid absorbent material.

33. The method of claim 31 wherein decontaminating is achieved by contacting the surface with liquid decontaminating solution.

34. The method of claim 33 wherein said liquid decontaminating solution is a disinfectant.

35. The method of claim 34 wherein the disinfectant is a solution of bleach.

36. The method of claim 31 wherein the instrument contains a filter cap assembly which is subjected to decontamination.

37. The method of claim 31 wherein after step d) the remainder of the second sample in the second sample vial is subjected to nucleic acid amplification.

38. The method of claim 31 wherein the sample is selected from the group consisting of a blood containing sample, a cervical cells containing sample, and a fine needle biopsy containing sample.
